(19) United States
(12) Patent Application Publication
(10) Pub. No.: US 2008/0176919 A1
(43) Pub. Date: Jul. 24, 2008

(54) Imidazole Derivatives As Enzyme Reverse Transcriptase Modulators

(75) Inventors: Lyn Howard Jones, Kent (GB); Sandra Dora Newman, Kent (GB); Nigel Alan Swain, Kent (GB)

Correspondence Address: PFIZER INC;Steve T. Zelson, 150 EAST 42ND STREET, 5TH FLOOR - STOP 49, NEW YORK, NY, 10017-5612, US

(73) Assignee: Pfizer Inc

(21) Appl. No.: 11/813,063

(22) PCT Filed: Dec. 23, 2005
(86) PCT No.: PCT/IB2005/003994

§ 371 Date: Jun. 28, 2007

Related U.S. Application Data
(60) Provisional application No. 60/645,200, filed on Jan. 18, 2005.

(30) Foreign Application Priority Data
Jan. 6, 2005 (GB) 0500195.3

(51) Int. Cl. A61K 31/4164; C07D 233/84; A61P 31/18

(52) U.S. Cl. 514/398; 548/323.5; 548/324.5; 548/324.1

(57) ABSTRACT

This invention relates to benzonitrile derivatives of formula (I) or pharmaceutically acceptable salts, solvates or derivative thereof, wherein R₁ to R₃ are defined in the description, to their use in medicine, and to compositions containing them. The compounds of the present invention bind to the enzyme reverse transcriptase and are modulators, especially inhibitors thereof.

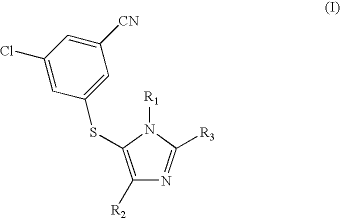

This invention relates to benzonitrile derivatives, to their use in medicine, to compositions containing them, to processes for their preparation and to intermediates used in such processes.

The compounds of the present invention bind to the enzyme reverse transcriptase and are modulators, especially inhibitors thereof. Reverse transcriptase is implicated in the infectious lifecycle of HIV, and compounds which interfere with the function of this enzyme have shown utility in the treatment of conditions including AIDS. There is a constant need to provide new and better modulators, especially inhibitors, of HIV reverse transcriptase since the virus is able to mutate, becoming resistant to the effects of known modulators.

According to the present invention there is provided a compound of formula (I):

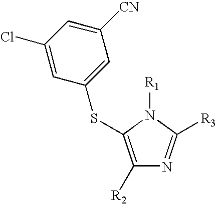

or a pharmaceutically acceptable salt or solvate or derivative thereof, wherein:

R₁ is (C₁-C₄)alkyl or (C₃-C₆)cycloalkyl, wherein said alkyl is optionally substituted by pyridyl or pyridyl N-oxide;

R₂ is (C₁-C₄)alkyl, (C₃-C₆)cycloalkyl, or trifluoromethyl;

R₃ is —(CH₂)ₘOH, —(CH₂)ₘOC(O)NR₄R₅, —(CH₂)ₘNR₄R₅, or —(CH₂)ₘ NHC(O)NR₄R₅;

R₄ and R₅ independently are H or (C₁-C₄)alkyl;

m is 1, 2, 3 or 4.

The term “alkyl” refers to a straight-chain or branched-chain saturated aliphatic hydrocarbon radical containing the specified number of carbon atoms. Examples of alkyl radicals include, but are not limited to, methyl, ethyl, n-propyl, isopropyl, n-butyl, isobutyl, sec-butyl, tert-butyl.

The term “cycloalkyl” refers to a carbocyclic ring composed of 3-6 carbons. Examples of carbocyclic rings include cyclopropyl, cyclobutyl, cyclopentyl, cyclohexyl.

In one embodiment, R₁ is (C₁-C₄)alkyl.

In one embodiment, R₂ is (C₁-C₄)alkyl or trifluoromethyl.

In one embodiment, R₃ is —(CH₂)ₘOH, —(CH₂)ₘOC(O)NR₄R₅, or —(CH₂)ₘNR₄R₅. In a further embodiment, R₃ is —(CH₂)ₘOH or —(CH₂)ₘOC(O)NR₄R₅

In one embodiment, R₄ and R₅ are H.

In one embodiment, m is 1 or 2.

It is to be understood that the invention covers all combinations of particular embodiments of the invention as described hereinabove, consistent with the definition of compounds of formula (I).

Pharmaceutically acceptable salts of the compounds of formula (I) include the acid addition and base salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002).

Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I) or by ring-opening a suitable cyclic precursor, for example, a lactone or lactam, using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004). For a general review of multi-component complexes, see J Pharm Sci, 64 (8), 1269-1288, by Haleblian (August 1975).

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N—N⁺(CH₃)₃) polar head group. For more information, see Crystals and the Polarizing Microscope by N. H. Hartshorne and A. Stuart, 4^{th} Edition (Edward Arnold, 1970).

Hereinafter all references to compounds of formula (I) include references to salts, solvates, multi-component complexes and liquid crystals thereof and to solvates, multi-component complexes and liquid crystals of salts thereof.

The compounds of the invention include compounds of formula (I) as hereinbefore defined, including all polymorphs and crystal habits thereof, prodrugs and isomers thereof (including optical, geometric and tautomeric isomers) as hereinafter defined and isotopically-labeled compounds of formula (I).

As indicated, so-called ‘prodrugs’ of the compounds of formula (I) are also within the scope of the invention. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in Design of Prodrugs by H. Bundgaard (Elsevier, 1985).

Some examples of prodrugs in accordance with the invention include
(i) where the compound of formula (I) contains a carboxylic acid functionality (—COOH), an ester thereof, for example, a compound wherein the hydrogen of the carboxylic acid functionality of the compound of formula (I) is replaced by (C₁-C₈)alkyl;
(ii) where the compound of formula (I) contains an alcohol functionality (—OH), an ether thereof, for example, a compound wherein the hydrogen of the alcohol functionality of the compound of formula (I) is replaced by (C₁-C₆)alkanoyloxymethyl; and
(iii) where the compound of formula (I) contains a primary or secondary amino functionality (—NH₂ or —NHR where R≠H), an amide thereof, for example, a compound wherein, as the case may be, one or both hydrogens of the amino functionality of the compound of formula (I) is/are replaced by (C₁-C₁₀)alkanoyl.

Further examples of replacement groups in accordance with the foregoing examples and examples of other prodrug types may be found in the aforementioned references.

Moreover, certain compounds of formula (I) may themselves act as prodrugs of other compounds of formula (I).

Also included within the scope of the invention are metabolites of compounds of formula (I), that is, compounds formed in vivo upon administration of the drug. Some examples of metabolites in accordance with the invention include
(i) where the compound of formula (I) contains a methyl group, an hydroxymethyl derivative thereof (—CH₃->-CH₂OH):
(ii) where the compound of formula (I) contains an alkoxy group, an hydroxy derivative thereof (—OR³->-OH);
(iii) where the compound of formula (I) contains a tertiary amino group, a secondary amino derivative thereof (—NR¹R²->—NHR¹ or —NHR²);
(iv) where the compound of formula (I) contains a secondary amino group, a primary derivative thereof (—NHR¹->—NH₂);
(v) where the compound of formula (I) contains a phenyl moiety, a phenol derivative thereof (-Ph->-PhOH); and
(vi) where the compound of formula (I) contains an amide group, a carboxylic acid derivative thereof (—CONH₂->COOH).

Compounds of formula (I) containing one or more asymmetric carbon atoms can exist as two or more stereoisomers. Where a compound of formula (I) contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of formula (I) containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of formula (I), including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

The present invention includes all pharmaceutically acceptable isotopically-labeled compounds of formula (I) wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and 125I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labeled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Representative compounds of formula (I) include the compounds of examples 1, 3 and 8, and pharmaceutically acceptable salts, solvates or derivatives thereof.

Compounds of formula (I) may be prepared by any methods known for the preparation of compounds of analogous structure.

Compounds of formula (I), and intermediates thereto, may be prepared according to the schemes that follow.

In these schemes: X is halo and preferably chloro; Y is halo and preferably iodo; THF means tetrahydrofuran; DMSO means dimethyl sulphoxide; DCM means dichloromethane; DMF means N,N-dimethylformamide; MeCN means acetonitrile; NMP means 1-methyl-2-pyrrolidinone; LDA means lithium diisopropylamide; MeOH means methanol; EtOH means ethanol; 0.88 SG means concentrated ammonium hydroxide solution, 0.88 ammonia; rt means room temperature; eq. means equivalent.

It will be appreciated by those skilled in the art that certain of the procedures described in the schemes for the preparation of compounds of formula (I) or intermediates thereto may not be applicable to some of the possible substituents.

It will be further appreciated by those skilled in the art that it may be necessary or desirable to carry out the transformations described in the schemes in a different order from that described, or to modify one or more of the transformations, to provide the desired compound of formula (I).

Compounds of formula (I) may be prepared as shown in scheme 1.

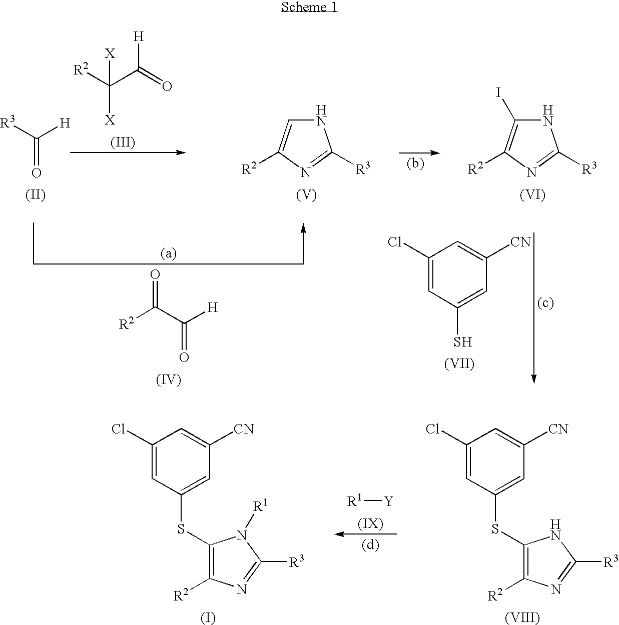

Compounds of general formula (II) are either commercially available or can be prepared as described in Tetrahedron, 56, 5303-5310; 2000.

Compounds of general formula (III) are either commercially available or can be prepared as described in Synthesis, 455-456, 1975.

Compounds of formula (IV) are either commercially available or can be prepared by analogy with the methods of Baldwin and Kasinger (J. Med. Chem. 18(9) 895-900; 1975). Compound (IV) is typically prepared by reaction of 1.0 eq. of ketone R²C(O)CHZ^{a}Z^{b}, preferably where Z^{a}=Z^{b}=bromo, with 2.0 eq. sodium acetate trihydrate, in a suitable solvent such as water, heated under reflux for 0.5-1 h.

### (a) Cyclisation

Compounds of formula (V) may be prepared by the reaction of a compound of formula (II) with a compound of formula (III), where X is halo and preferably chloro, in the presence of a source of ammonia, such as concentrated ammonium hydroxide solution, 0.88 SG or ammonium acetate, in a suitable solvent such as MeCN, at rt for 18-48 h. Typical conditions comprise of 1.0 eq. of compound (II), 1.0 eq. of compound (III) and excess 0.88 ammonia, in MeCN at rt for 48 h.

Compounds of formula (V) may alternatively be prepared by cyclisation of compounds (II) and (IV) in the presence of a source of ammonia, such as concentrated ammonium hydroxide solution, 0.88 SG or ammonium acetate, in a suitable solvent such as MeOH or THF, at rt for 18-48 h. Typical conditions comprise of 1.0 eq. of compound (II), 1.1 eq. of compound (IV) and excess 0.88 ammonia, in MeOH, at rt for 18 h.

### (b) Iodination

Compounds of formula (VI) may be prepared by the iodination of a compound of formula (V) using a source of iodine, such as molecular iodine, iodine with periodic acid dihydrate or N-iodosuccinimide, optionally in the presence of a suitable base such as sodium hydroxide or potassium hydroxide, in a suitable solvent such as DCM, MeOH or a biphasic system such as chloroform and acetic acid, at a temperature between 0° C. to 60° C., for 0.5 to 4 h. Typical conditions comprise of 1.0 eq. of compound (V), 1.0-1.5 eq. of iodine and 1.0 eq. periodic acid dihydrate in a mixture of chloroform and acetic acid, heated at 60° C. for 4 h, or, 1.0 eq. of compound (V), 1-1.5 eq. of base such as sodium hydroxide and 1-1.3 eq. of iodine in a mixture of DCM and MeOH, at 0° C. for 0.5-1.0 h.

Alternatively, compounds of formula (VI) may be prepared from compounds of formula (II) and (III) by combination of steps (a) and (b) in a ‘one pot’ synthesis. Typical conditions comprise of

a) 1.0 eq. of compound (II), 1.0 equivalent of (III) and excess 0.88 ammonia, in MeCN at rt for 16 h;
b) 1-1.5 eq. of base such as sodium hydroxide and 1-1.3 eq. of iodine in a mixture of DCM and MeOH, at 0° C. for 1 h.

### (c) Nucleophilic Substitution

Compounds of formula (VIII) may be prepared by the reaction of compounds of formula (VI) and compounds of formula (VII) under conventional conditions. Conveniently, the reaction may be effected using a base, such as an alkali metal base, for example, an alkali metal carbonate (e.g., potassium, sodium or caesium carbonate); optionally in the presence of copper (I) iodide, in a suitable solvent such as a polar aprotic solvent (e.g., MeCN or DMF), optionally at elevated temperature for 1-24 h. Typical conditions comprise of 1.0 eq. of compound (VI), 1.0-1.3 eq. of compound (VII), 1.1-1.5 eq. of caesium carbonate, optionally in the presence of copper (I) iodide (cat.), in MeCN, at reflux for 1-24 h.

### (d) Alkylation

Compounds of formula (I) may be prepared by alkylating a compound of formula (VIII) with a compound of formula (IX) under conventional alkylating conditions. Conveniently, alkylation is effected using a base, such as an alkali metal base, for example, an alkali metal carbonate (e.g., sodium, potassium or caesium carbonate), in the presence of a solvent, such as a polar aprotic solvent (e.g., MeCN or DMF), at rt for 18 hours. Typical conditions comprise of 1.0 eq. of compound (VIII), 1.0-1.2 eq. of compound (IX), 1.5-2.0 eq. of potassium carbonate, in DMF at rt for 18 h.

Alternatively, compounds of formula (I) may be prepared as described in Scheme 2.

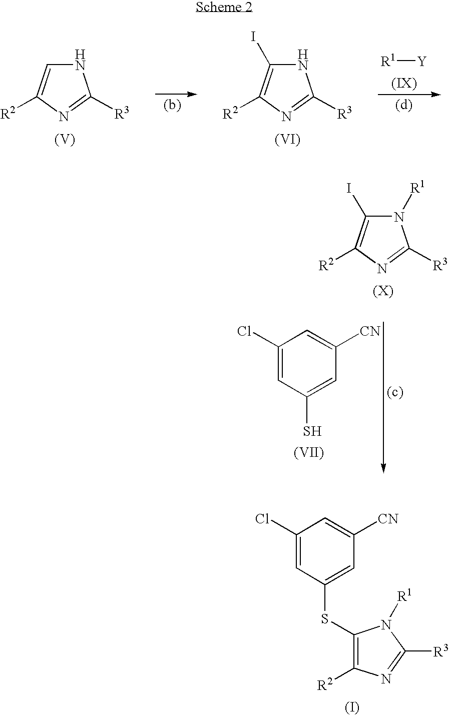

(b) Iodination Compounds of formula (VI) may be prepared by the iodination of compounds of formula (V), as described in scheme 1.

### (d) Alkylation

Compounds of formula (X) may be prepared by alkylation of compounds of formula (VI) with compound (IX), as described in scheme 1.

(c) Nucleophilic substitution

Compounds of formula (I) may be prepared by reaction of compound (X) with compounds (VII), as described in scheme 1.

Compounds of formula (VII) may be prepared as shown in scheme 3.

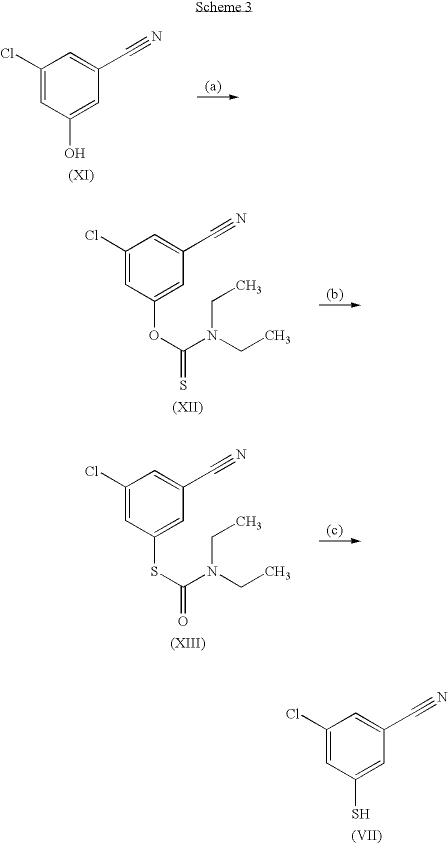

### (a) Nucleophilic Substitution

Compounds of formula (XII) can be prepared by treatment of compounds of formula (XI) with a suitable base such as sodium hydride or LDA, followed by quench of the intermediate anion with diethylthiocarbamoyl chloride, in a suitable solvent such as NMP or DMSO, at ambient to elevated temperature for 2-4-h. Typical conditions comprise of 1.0 eq. of compound (XI), 1.3 eq. sodium hydride (60% dispersion in mineral oil) and 1.3 eq. of diethylthiocarbamoyl chloride in NMP, at a temperature between 25-75° C. for 2.5 h.

### (b) Rearrangement

Compounds of formula (XIII) can be prepared from compounds of formula (XII) by heating compound (XII) at elevated temperature for 18-24 hours. Typical conditions comprise of direct heating of compound (XII) at a temperature between 180-200° C. for 22 h.

### (c) De-Protection

Compounds of formula (VII) can be prepared from compounds of formula (XIII) using conventional methods. See for example, those described in ‘Protective Groups in Organic Synthesis’ by Theodora W Green and Peter G M Wuts, third edition, (John Wiley and Sons, 1999), in particular chapter 6. Typical conditions comprise of 1.0 eq. of compound (XIII) and 1.0 eq. of sodium hydroxide in MeOH, under ambient conditions for 18-24 hours.

Compounds of formula (XI) may be prepared as described in WO2004031178, p 27.

It will be appreciated by those skilled in the art that it may be necessary or desirable at any stage in the synthesis of compounds of formula (I) to protect one or more sensitive groups in the molecule so as to prevent undesirable side reactions. In particular, it may be necessary or desirable to protect amino or hydroxy groups. The protecting groups used in the preparation of compounds of formula (I) may be used in conventional manner. See, for example, those described in ‘Protective Groups in Organic Synthesis’ by Theodora W Green and Peter G M Wuts, third edition, (John Wiley and Sons, 1999), in particular chapter 2, pages 17-245 (“Protection for the Hydroxyl Group”), and chapter 7, pages 494-653 (“Protection for the Amino Group”), incorporated herein by reference. Removal of such groups can also be achieved using conventional methods as described above.

For example, when R³ contains a hydroxyl group, compounds of formula (I) may be prepared by cleavage of a benzyl protecting group using 2M boron trichloride dimethylsulfide complex solution in DCM, under ambient conditions (e.g. examples 1 and 2).

When R³ incorporates an amino group, compounds of formula (I) may be prepared by removal of a phthalimide protecting group using hydrazine monohydrate, in a suitable solvent such as EtOH, at 45° C. for 18 h (e.g. examples 4 and 5).

It will be further appreciated that compounds of formula (I) may also be converted to alternative compounds of formula (I) using standard chemical reactions and transformations. For example, when R³ is hydroxy, a carbamic acid is afforded by reaction with trichloroacetylisocyanate (e.g. examples 6 and 8).

According to another aspect, the invention provides a process for preparing compounds of formula (I) comprising reaction of a compound of formula (VIII) with a compound of formula (IX) or reaction of a compound of formula (X) with a compound of formula (VII).

Also within the scope of the invention are intermediate compounds of formulae (VII), (VIII), (XII) and (XIII) as hereinbefore defined, all salts, solvates and complexes thereof and all solvates and complexes of salts thereof as defined hereinbefore for compounds of formula (I). The invention includes all polymorphs of the aforementioned species and crystal habits thereof.

When preparing compounds of formula (I) in accordance with the invention, it is open to a person skilled in the art to routinely select the form of compound of formula (VII), (VIII), (XII) or (XIII) which provides the best combination of features for this purpose. Such features include the melting point, solubility, processability and yield of the intermediate form and the resulting ease with which the product may be purified on isolation.

The compounds of the invention are reverse transcriptase inhibitors and are therefore of use in the treatment of a HIV, a retroviral infection genetically related to HIV, and AIDS.

Accordingly, in another aspect the invention provides a compound of the formula (I) or a pharmaceutically acceptable salt, solvate or derivative thereof for use as a medicament.

In another aspect, the invention provides a compound of the formula (I) or a pharmaceutically acceptable salt, solvate or derivative thereof for use as a reverse transcriptase inhibitor or modulator.

In another aspect the invention provides a compound of the formula (I) or a pharmaceutically acceptable salt, solvate or derivative thereof for use in the treatment of a HIV, a retroviral infection genetically related to HIV, or AIDS.

In another aspect, the invention provides the use of a compound of the formula (I) or a pharmaceutically acceptable salt, solvate or derivative thereof in the manufacture of a medicament having reverse transcriptase inhibitory or modulating activity.

In another aspect the invention provides the use of a compound of the formula (I) or of a pharmaceutically acceptable salt, solvate or derivative thereof in the manufacture of a medicament for the treatment of a HIV, a retroviral infection genetically related to HIV, or AIDS.

In another aspect, the invention provides a method of treatment of a mammal, including a human being, with a reverse transcriptase inhibitor or modulator, which comprises treating said mammal with an effective amount of a compound of the formula (I) or a pharmaceutically acceptable salt, solvate or derivative thereof.

In another aspect the invention provides a method of treatment of a mammal, including a human being, with a HIV, a retroviral infection genetically related to HIV, or AIDS, which comprises treating said mammal with an effective amount of a compound of formula (I) or a pharmaceutically acceptable salt, solvate or derivative thereof.

The compounds of formula (I) should be assessed for their biopharmaceutical properties, such as solubility and solution stability (across pH), permeability, etc., in order to select the most appropriate dosage form and route of administration for treatment of the proposed indication.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

The compounds of the invention may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function.

The compound of formula (I) may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula (I) may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(dl-lactic-coglycolic)acid (PGLA) microspheres.

The compounds of the invention may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, gels, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to human patients, the total daily dose of the compounds of the invention is typically in the range 1 to 10000 mg, such as 10 to 1000 mg, for example 25 to 500 mg, depending, of course, on the mode of administration. The total daily dose may be administered in single or divided doses and may, at the physician's discretion, fall outside of the typical range given herein.

These dosages are based on an average human subject having a weight of about 60 kg to 70 kg. The physician will readily be able to determine doses for subjects whose weight falls outside this range, such as infants and the elderly.

For the avoidance of doubt, references herein to “treatment” include references to curative, palliative and prophylactic treatment.

Accordingly in another aspect the invention provides a pharmaceutical composition including a compound of the formula (I) or a pharmaceutically acceptable salt, solvate or derivative thereof together with one or more pharmaceutically acceptable excipients, diluents or carriers.

The compounds of formula (I) and their pharmaceutically acceptable salts, solvates and derivatives have the advantage that they are more selective, have a more rapid onset of action, are more potent, are better absorbed, are more stable, are more resistant to metabolism, have a reduced ‘food effect’, have an improved safety profile or have other more desirable properties (e.g. with respect to solubility or hygroscopicity) than the compounds of the prior art.

In particular, the compounds of formula (I) are more resistant to metabolism. In providing compounds of formula (I) which exhibit increased resistance to metabolism coupled with comparable or improved potency, the invention provides compounds which are therapeutically effective NNRT is at significantly lower dosages than the compounds of the prior art. Moreover, the increased solubility of compounds of formula (I) further facilitates lower dosages and flexibility in the routes of administration. These advantages can be expected to improve efficacy, safety, and patient compliance during treatment; and reduce the cost thereof.

The compounds of formula (I) and their pharmaceutically acceptable salts, solvates and derivatives may be administered alone or as part of a combination therapy. Thus included within the scope of the present invention are embodiments comprising coadministration of, and compositions which contain, in addition to a compound of the invention, one or more additional therapeutic agents. Such multiple drug regimens, often referred to as combination therapy, may be used in the treatment and prevention of infection by human immunodeficiency virus, HIV. The use of such combination therapy is especially pertinent with respect to the treatment and prevention of infection and multiplication of the human immunodeficiency virus, HIV, and related pathogenic retroviruses within a patient in need of treatment or one at risk of becoming such a patient. The ability of such retroviral pathogens to evolve within a relatively short period of time into strains resistant to any monotherapy which has been administered to said patient is well known in the literature. A recommended treatment for HIV is a combination drug treatment called Highly Active Anti-Retroviral Therapy, or HAART. HAART combines three or more HIV drugs. Thus, the methods of treatment and pharmaceutical compositions of the present invention may employ a compound of the invention in the form of monotherapy, but said methods and compositions may also be used in the form of combination therapy in which one or more compounds of the invention are coadministered in combination with one or more additional therapeutic agents such as those described in detail further herein.

In a further embodiment of the invention, combinations of the present invention include treatment with a compound of formula (I), or a pharmaceutically acceptable salt, solvate or derivative thereof, and one or more additional therapeutic agents selected from the following: HIV protease inhibitors (PIs), including but not limited to indinavir, ritonavir, saquinavir, nelfinavir, lopinavir, amprenavir, atazanavir, tipranavir, AG1859 and TMC 114; non-nucleoside reverse transcriptase inhibitors (NNRTIs), including but not limited to nevirapine, delavirdine, capravirine, efavirenz, GW-8248, GW-5634 and etravirine; nucleoside/nucleotide reverse transcriptase inhibitors, including but not limited to zidovudine, didanosine, zalcitabine, stavudine, lamivudine, abacavir, adefovir dipivoxil, tenofovir and emtricitabine; CCR5 antagonists, including but not limited to:
N-{(1S)-3-[3-(3-isopropyl-5-methyl-4H-1,2,4-triazol-4-yl)-exo8-azabicyclo[3.2.1]oct-8-yl]-1-phenylpropyl}-4,4-difluorocyclohexanecarboxamide or a pharmaceutically acceptable salt, solvate or derivative thereof,
methyl 1-endo{8-[(3S)-3-(acetylamino)-3-(3-fluorophenyl)propyl]-8-azabicyclo[3.2.1]oct-3-yl}-2-methyl-1,4,6,7-tetrahydro-5H-imidazo[4,5-c]pyridine-5-carboxylate or a pharmaceutically acceptable salt, solvate or derivative thereof,
ethyl 1-endo{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate or a pharmaceutically acceptable salt, solvate or derivative thereof, Sch-D, ONO-4128, AMD-887, GW-873140 and CMPD-167; CXCR4 antagonists, including but not limited to AMD-3100, AMD-070, and KRK-2731; integrase inhibitors, including but not limited to L-870,810; entry (e.g. fusion) inhibitors, including but not limited to enfuviritide; agents which inhibit the interaction of gp120 and CD4, including but not limited to BMS806 and BMS-488043; and RNaseH inhibitors.

There is also included within the scope the present invention, combinations of a compound of formula (I), or a pharmaceutically acceptable salt, solvate or derivative thereof, together with one or more additional therapeutic agents independently selected from the group consisting of proliferation inhibitors, e.g. hydroxyurea; immunomodulators, such as granulocyte macrophage colony stimulating growth factors (e.g. sargramostim), and various forms of interferon or interferon derivatives; other chemokine receptor agonists/antagonists such as CXCR4 antagonists, e.g. AMD-3100, AMD-070 or KRK-2731; tachykinin receptor modulators (e.g. NK1 antagonists) and various forms of interferon or interferon derivatives; inhibitors of viral transcription and RNA replication; agents which influence, in particular down regulate, CCR5 receptor expression; chemokines that induce CCR5 receptor internalisation such as MIP-1α, MIP-1β, RANTES and derivatives thereof; and other agents that inhibit viral infection or improve the condition or outcome of HIV-infected individuals through different mechanisms.

Agents which influence (in particular down regulate) CCR5 receptor expression include immunosupressants, such as calcineurin inhibitors (e.g. tacrolimus and cyclosporin A); steroids; agents which interfere with cytokine production or signalling, such as Janus Kinase (JAK) inhibitors (e.g. JAK-3 inhibitors, including 3-{(3R,4R)-4-methyl-3-[methyl-(7H-pyrrolo[2,3-d]pyrimidin-4-yl)-amino]-piperidin-1-yl}-3-oxo-propionitrile) and pharmaceutically acceptable salts, solvates or derivatives thereof; cytokine antibodies (e.g. antibodies that inhibit the interleukin-2 (IL-2) receptor, including basiliximab and daclizumab); and agents which interfere with cell activation or cell cycling, such as rapamycin.

There is also included within the scope the present invention, combinations of a compound of formula (I), or a pharmaceutically acceptable salt, solvate or derivative thereof, together with one or more additional therapeutic agents which yet further slow down the rate of metabolism of the compound of the invention, thereby leading to increased exposure in patients. Increasing the exposure in such a manner is known as boosting. This has the benefit of increasing the efficacy of the compound of the invention or reducing the dose required to achieve the same efficacy as an unboosted dose. The metabolism of the compounds of the invention includes oxidative processes carried out by P450 (CYP450) enzymes, particularly CYP 3A4 and conjugation by UDP glucuronosyl transferase and sulphating enzymes. Thus, among the agents that may be used to increase the exposure of a patient to a compound of the present invention are those that can act as inhibitors of at least one isoform of the cytochrome P450 (CYP450) enzymes. The isoforms of CYP450 that may be beneficially inhibited include, but are not limited to, CYP1A2, CYP2D6, CYP2C9, CYP2C19 and CYP3A4. Suitable agents that may be used to inhibit CYP 3A4 include, but are not limited to, ritonavir, saquinavir or ketoconazole.

It will be appreciated by a person skilled in the art, that a combination drug treatment, as described herein above, may comprise two or more compounds having the same, or different, mechanism of action. Thus, by way of illustration only, a combination may comprise a compound of the invention and: one or more other NNRTIs; one or more NRTIs and a PI; one or more NRTIs and a CCR5 antagonist; a PI; a PI and an NNRTI; and so on.

In addition to the requirement of therapeutic efficacy, which may necessitate the use of therapeutic agents in addition to the compounds of the invention, there may be additional rationales which compel or highly recommend the use of a combination of a compound of the invention and another therapeutic agent, such as in the treatment of diseases or conditions which directly result from or indirectly accompany the basic or underlying disease or condition. For example, it may be necessary or at least desirable to treat Hepatitis C Virus (HCV), Hepatitis B Virus (HBV), Human Papillomavirus (HPV), opportunistic infections (including bacterial and fungal infections), neoplasms, and other conditions which occur as the result of the immune-compromised state of the patient being treated. Other therapeutic agents may be used with the compounds of the invention, e.g., in order to provide immune stimulation or to treat pain and inflammation which accompany the initial and fundamental HIV infection.

Accordingly, therapeutic agents for use in combination with the compounds of formula (I) and their pharmaceutically acceptable salts, solvates and derivatives also include: interferons, pegylated interferons (e.g. peginterferon alfa-2a and peginterferon alfa-2b), lamivudine, ribavirin, and emtricitabine for the treatment of hepatitis; antifungals such as fluconazole, itraconazole, and voriconazole; antibacterials such as azithromycin and clarithromycin; interferons, daunorubicin, doxorubicin, and paclitaxel for the treatment of AIDS related Kaposi's sarcoma; and cidofovir, fomivirsen, foscarnet, ganciclovir and valcyte for the treatment of cytomegalovirus (CMV) retinitis.

Further combinations for use according to the invention include combination of a compound of formula (I), or a pharmaceutically acceptable salt, solvate or derivative thereof with a CCR1 antagonist, such as BX-471; a beta adrenoceptor agonist, such as salmeterol; a corticosteroid agonist, such fluticasone propionate; a LTD4 antagonist, such as montelukast; a muscarinic antagonist, such as tiotropium bromide; a PDE4 inhibitor, such as cilomilast or roflumilast; a COX-2 inhibitor, such as celecoxib, valdecoxib or rofecoxib; an alpha-2-delta ligand, such as gabapentin or pregabalin; a beta-interferon, such as REBIF; a TNF receptor modulator, such as a TNF-alpha inhibitor (e.g. adalimumab); a HMG CoA reductase inhibitor, such as a statin (e.g. atorvastatin); or an immunosuppressant, such as cyclosporin or a macrolide such as tacrolimus.

In the above-described combinations, the compound of formula (I) or a pharmaceutically acceptable salt, solvate or derivative thereof and other therapeutic agent(s) may be administered, in terms of dosage forms, either separately or in conjunction with each other; and in terms of their time of administration, either simultaneously or sequentially. Thus, the administration of one component agent may be prior to, concurrent with, or subsequent to the administration of the other component agent(s).

Accordingly, in a further aspect the invention provides a pharmaceutical composition comprising a compound of formula (I) or a pharmaceutically acceptable salt, solvate or derivative thereof and one or more additional therapeutic agents.

It is to be appreciated that all references herein to treatment include curative, palliative and prophylactic treatment.

The invention is illustrated by the following Examples and Preparations in which the following further abbreviations may be used:

EtOAc means ethyl acetate; AcOH means acetic acid, NMR means nuclear magnetic resonance; LRMS means low resolution mass spectrum; HRMS means high resolution mass spectrum; APCI means atmospheric pressure chemical ionisation; tlc means thin layer chromatography.

### Preparation 1

### 2-(2-Benzyloxy-ethyl)-5-trifluoromethyl-1H-imidazole

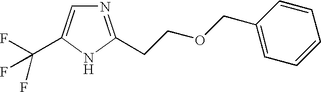

A mixture of sodium acetate trihydrate (2.7 g, 20 mmol) and 1-dibromo-3,3,3-trifluoroacetone (2.7 g, 10 mmol) in water (18 mL) was heated under reflux for 30 min. The mixture was then cooled to rt and was slowly added to a solution of 4-(phenylmethoxy)propanal (Tetrahedron, 56, 5303-5310; 2000), (1.48 g, 9 mmol) and concentrated ammonium hydroxide solution (11 mL) in MeOH (45 mL). The mixture was stirred at rt for 18 h and was then evaporated under reduced pressure. The aqueous residue was extracted with EtOAc (3×50 mL) and the combined organic solution was dried over magnesium sulfate and concentrated in vacuo to give an oil. The oil was then triturated in water with a trace of MeOH to afford the title compound as a crystalline solid in 88% yield (2.4 g).

¹H NMR (400 MHz, CDCl₃) δ: 3.08 (t, 2H), 3.80 (t, 2H), 4.75 (s, 2H), 7.22 (d, 1H), 7.30 (m, 5H) LRMS: m/z APCI-271 [M+H]⁺

### Preparation 2

### 2-(2-Benzyloxy-ethyl)-4-iodo-5-trifluoromethyl-1H-imidazole

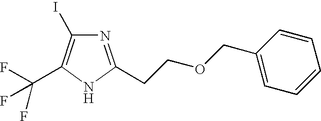

Iodine (5.6 g, 22 mmol), periodic acid dihydrate (4.6 g, 20 mmol) and chloroform (35 mL) were added to a solution of the compound of preparation 1 (5.4 g, 20 mmol) in AcOH (105 mL), and the mixture was stirred at 50° C. for 2 h and then at rt for 18 h. The reaction mixture was poured onto ice-cold 10% aqueous sodium bisulphite solution and was extracted with EtOAc (3×100 mL). The combined organic solution was dried over magnesium sulphate and concentrated in vacuo. The residue was azeotroped with toluene and purified by column chromatography on silica gel, eluting with ethyl acetate:pentane, 33:66, to 50:50 to afford the title compound as a pale yellow oil in 49% yield (3.9 g).

¹H NMR (400 MHz, CDCl₃) δ: 3.05 (m, 2H), 3.78 (t, 2H), 4.58 (s, 2H), 7.38 (m, 5H). LRMS: m/z APCI-397[M+H]⁺

### Preparation 3

### 2-[2-(Benzyloxy)ethyl]-4-ethyl-5-iodo-1H-imidazole

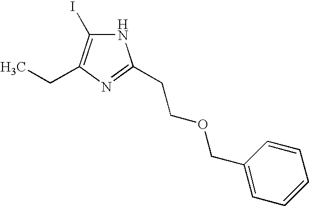

A solution of 3-benzyloxy-1-propionaldehyde (Tetrahedron, 2000, 56, 5303-5310) (135 g, 957 mmol) and 2,2-dichlorobutanal (Synthesis, 1975, 455-456) (154.3 g, 957 mmol) in MeCN (250 mL) was cooled to −5° C. and treated with 0.88 ammonia (650 mL, added in 50 mL portions). The reaction was then allowed to warm to rt and stirred for 16 h. DCM (500 mL) was added to the mixture and the layers separated. The aqueous layer was further extracted with DCM (2×200 mL) and the combined organic fraction was washed with brine (500 mL), dried over magnesium sulfate and evaporated under reduced pressure to give 244 g of a thick orange oil. This oil was dissolved in DCM (400 mL), cooled to 0° C. and treated with a solution of sodium hydroxide (46.61 g, 1.17 mol) in water (200 mL). A slurry of iodine (295.8 g, 1.17 mol) in methanol:dichloromethane (1:1, 400 mL) was added and the resulting brown-black mixture was stirred at 0° C. for 1 h, then allowed to warm to 8° C. The mixture was diluted with DCM (400 mL) and treated with 10% aqueous sodium sulphite solution (500 mL) with vigorous stirring. The layers were separated and the aqueous layer further extracted with DCM (2×300 mL). The combined organic solution was washed with 10% aqueous sodium sulphite solution (500 mL) and brine (600 mL), dried over magnesium sulfate and concentrated in vacuo. The residue was purified by column chromatography on silica gel, eluting with pentane:ethyl acetate, 50:50 to 0:100, to give a solid. This solid was triturated with pentane to afford the title compound as a white solid in 34% yield (117.44 g).

LRMS: m/z APCI-357 [M+H]⁺

### Preparation 4

### Diethyl-thiocarbamic acid O-(3-chloro-5-cyano-phenyl) ester

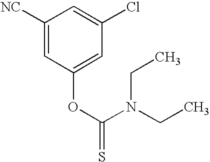

A solution of 3-chloro-5-hydroxybenzonitrile [(10.1 g, 66 mmol) WO2004031178, p27] in NMP (40 mL) was added to an ice-cooled slurry of sodium hydride (60% dispersion in mineral oil, 3.42 g, 85 mmol) in NMP (30 mL). The mixture was allowed to warm to rt and was stirred for 30 min. A solution of diethylthiocarbamoyl chloride (12.97 g, 85 mmol) in NMP (50 mL) was then added and the mixture was stirred for 30 min at rt and at 75° C. for 2 h. The cooled mixture was diluted with water (300 mL), extracted with EtOAc (3×200 mL) and the combined organic solution was washed with brine, dried over magnesium sulfate and concentrated in vacuo to give a red oil. The oil was purified by column chromatography on silica gel, eluting with pentane:ethyl acetate, 100:0 to 80:20, and the relevant fractions were concentrated in vacuo. The residue was then re-crystallised from pentane:ethyl acetate, 90:10, to afford the title compound as a solid in 74% yield (13.12 g).

¹H NMR (400 MHz, CDCl₃) δ: 1.30 (m, 6H), 3.62 (q, 2H), 3.83 (q, 2H), 7.22 (s, 1H), 7.23 (s, 1H), 7.48 (s, 1H) LRMS: m/z APCI-269 [M+H]⁺, Microanalysis: C₁₂H₁₃ClN₂OS requires (%): C, 53.63; H, 4.88; N 10.42; found (%): C, 53.64; H, 4.83; N, 10.33.

### Preparation 5

### Diethyl-thiocarbamic acid S-(3-chloro-5-cyano-phenyl) ester

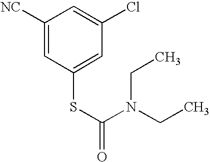

The compound of preparation 4 (13.2 g, 49 mmol) was heated between 180-200° C. for 12 h to give an orange oil. The oil was purified by column chromatography on silica gel, eluting with pentane:ethyl acetate, 100:0 to 20:80, to afford the title compound as a crystalline solid in 100% yield (13.2 g).

¹H NMR (400 MHz, CDCl₃) δ: 1.04-1.32 (bm, 6H), 3.40 (m, 2H), 3.83 (q, 2H), 7.60 (s, 1H), 7.68 (s, 1H), 7.72 (s, 1H) LRMS: m/z APCI-269 [M+H]⁺, Microanalysis: C₁₂H₁₃ClN₂OS requires (%): C, 53.63; H, 4.88; N, 10.42; found (%): C, 53.57; H, 4.80; N, 10.32.

### Preparation 6

### 3-Chloro-5-mercapto-benzonitrile

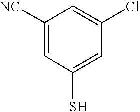

Sodium hydroxide (74 mg, 1.85 mmol) was added to a solution of the compound of preparation 5 (0.5 g, 1.86 mmol) in MeOH (2 mL) and the mixture was stirred at rt for 22 h. The reaction mixture was then concentrated in vacuo and the residue was diluted with 1M sodium hydroxide solution (5 mL) and washed with DCM (2×5 mL) and diethyl ether (5 mL). The aqueous solution was acidified with 2M hydrochloric acid and extracted with DCM (2×10 mL), diethyl ether (5 mL) and EtOAc (5 mL). The combined organic solution was washed with brine, dried over magnesium sulfate and concentrated in vacuo to afford the title compound in 82% yield (260 mg).

LRMS: m/z APCI 168 [M−H]⁻, Microanalysis: C₇H₄ClNS requires (%): C, 49.56; H, 2.38; N, 8.26; found (%): C, 49.44; H, 2.45; N, 8.25.

### Preparation 7

### 5-[2-(2-Benzyloxy-ethyl)-5-trifluoromethyl-3H-imidazol-4-ylsulfanyl]-3-chloro-benzonitrile

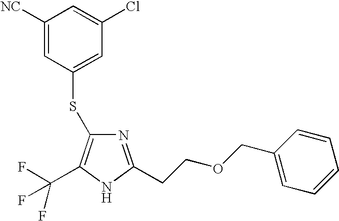

Caesium carbonate (0.89 g, 2.75 mmol) was added to a stirred solution of the compound of preparation 6 (0.43 g, 2.5 mmol) in MeCN (20 mL) and the mixture was stirred for 20 min at rt. The compound of preparation 2 (1 g, 2.5 mmol) was then added portionwise, followed by copper (I) iodide (95 mg, 0.5 mmol) and the reaction mixture was heated under reflux for 4 h. After this time, tlc analysis showed that starting material still remained and so further copper (I) iodide (45 mg, 0.24 mmol) was added to the mixture and heating continued for 18 h. The mixture was then cooled to rt and was concentrated in vacuo. The residue was partitioned between EtOAc and water and the resulting precipitate was filtered off. The layers of the filtrate were separated and the organic solution was dried over magnesium sulfate and concentrated in vacuo to give a dark green foam. Purification of the foam by column chromatography on silica gel, eluting with pentane:ethyl acetate, 75:25 to 67:33, afforded the title compound in 46% yield (500 mg).

¹H NMR (400 MHz, CDCl₃) δ: 3.10(m, 2H), 3.84 (t, 2H), 4.58 (s, 2H), 7.18 (s. 1H), 7.20-7.38 (m, 6H) 7.42(s, 1H). LRMS: m/z APCI-438 [M+H]⁺

### Preparation 8

### 5-[2-(2-Benzyloxy-ethyl)-5-ethyl-3H-imidazol-4-ylsulfanyl]-3-chloro-benzonitrile

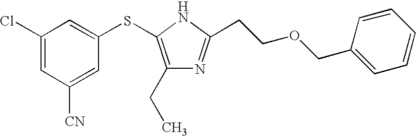

Caesium carbonate (1.47 g, 4.5 mmol) was added to a stirred solution of the compound of preparation 6 (0.7 g, 4.12 mmol) in MeCN (20 mL) and the mixture was stirred for 15 min at rt. A solution of the compound of preparation 3 (1.47 g, 4.12 mmol) in MeCN (20 mL) was then added and the reaction mixture was heated under reflux for 18 h. The mixture was then cooled to rt and was concentrated in vacuo. The residue was partitioned between EtOAc and water and the resulting precipitate was filtered off. The layers of the filtrate were separated and the aqueous solution was re-extracted with EtOAc. The combined organic solution was dried over magnesium sulfate and concentrated in vacuo to give a brown gum. Purification of the gum by column chromatography on silica gel, eluting with pentane:ethyl acetate, 75:25 to 50:50, followed by trituration with diethyl ether/pentane, afforded the title compound in 75% yield (1.2 g).

¹H NMR (400 MHz, CDCl₃) δ: 1.15(t, 3H), 2.63 (q, 2H), 3.09 (t, 2H), 3.85 (t, 2H), 4.63 (s, 2H), 7.15 (s, 1H), 7.35 (m, 7H), LRMS: m/z APCI-397 [M+H]⁺

### Preparation 9

### 5-[2-(2-Benzyloxy-ethyl)-3-ethyl-5-trifluoromethyl-3H-imidazol-4-ylsulfanyl]-3-chloro-benzonitrile

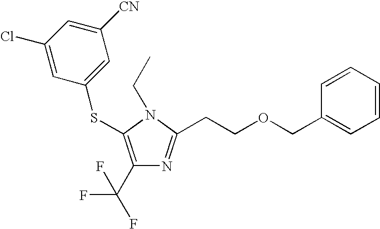

Potassium carbonate (208 mg, 1.5 mmol) was added to a solution of the compound of preparation 7 (440 mg, 1.0 mmol) in DMF (5 mL) and the mixture was stirred at rt for 10 min. Ethyl iodide (96 μL, 1.2 mmol) was then added dropwise to the mixture and stirring continued for 18 h. The mixture was then concentrated in vacuo and the residue was partitioned between EtOAc and water. The aqueous layer was separated and extracted with EtOAc, and the combined organic solution was dried over magnesium sulfate and concentrated in vacuo. The residue was purified by column chromatography on silica gel, eluting with toluene:ethyl acetate 75:25 to afford the title compound as a gum in 71% yield (330 mg).

¹H NMR (400 MHz, CDCl₃) δ: 1.18(t, 3H), 3.05 (t, 2H), 3.90 (t, 2H), 4.05 (q, 2H), 4.50 (s, 2H), 7.25 (m, 7H), 7.42 (s, 1H). HRMS: m/z found: 466.0962; C₂₂H₂₀ClF₃N₃OS requires 466.0960

### Preparation 10

### 5-[2-(2-Benzyloxy-ethyl)-3,5-diethyl-3H-imidazol-4-ylsulfanyl]-3-chloro-benzonitrile

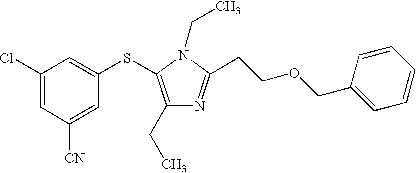

The title compound was prepared from the compound of preparation 8 and ethyl iodide, using a similar method to preparation 9 in 25% yield.

¹H NMR (400 MHz, CDCl₃) δ: 1.18(t, 3H), 1.22 (t, 3H), 2.65 (q, 2H), 3.10 (m, 2H), 3.95 (m, 4H), 4.50 (s, 2H), 7.20 (m, 8H) LRMS: m/z APCI-427 [M+H]⁺

### Preparation 11

### 2-Benzyloxymethyl-4-ethyl-1H-imidazole

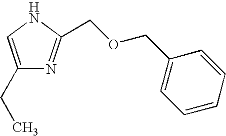

Benzyloxyacetaldehyde (11.4 mL, 80.9 mmol) was added to a stirred solution of 2,2-dichlorobutanal (Synthesis, 1975, 455-456) (11.4 g, 80.9 mmol) in MeCN (40 mL) at 0° C., followed by 0.88 ammonia (80 mL). The reaction was stirred at rt for 48 h. The mixture was then evaporated under reduced pressure and the residue was extracted with DCM (300 mL, 2×100 mL). The combined organic solutions were dried over magnesium sulfate, and concentrated in vacuo to give a dark brown oil. The oil was purified by chromatography on silica gel eluting with dichloromethane:methanol:0.88 ammonia, 100:0:0 to 95:5:0.5, followed by trituration with diethyl ether to afford the title compound as a pale brown solid in 53% yield (9.2 g).

¹H NMR (400 MHz, CDCl₃) δ: 1.23(t, 3H), 2.60 (q, 2H), 4.56 (s, 2H), 4.62 (s, 2H), 6.69 (s, 1H), 7.35 (m, 5H) LRMS: m/z APCI-217 [M+H]+Microanalysis: C₁₃H₁₆N₂O requires (%): C, 71.98; H, 7.44; N 12.85; found (%): C, 72.19; H, 7.48; N, 12.95.

### Preparation 12

### 2-Benzyloxymethyl-4-ethyl-5-iodo-1H-imidazole

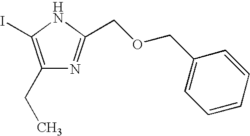

A solution of sodium hydroxide (1.88 g, 51.7 mmol) in water (25 mL) was added to an ice-cooled solution of the compound of preparation 11 (9.2 g, 42.5 mmol) in DCM (50 mL) and the mixture was stirred for 5 min. A solution of iodine (11.9 g, 47 mmol) in a mixture of DCM (80 mL) and MeOH (15 ml) was added dropwise over 30 min and the resulting mixture was stirred at 0° C. for 45 min. The reaction mixture was then diluted with DCM (200 mL), washed [sodium sulphite solution (100 mL), sodium bisulphite solution (100 mL) and then brine (200 mL)], dried over magnesium sulfate and concentrated in vacuo. Purification of the residue using an ISCO Companion® silica cartridge, eluting with pentane:ethyl acetate 80:20 to 67:33, afforded the title compound as a pale yellow gum in 65% yield (9.4 g).

¹H NMR (400 MHz, CDCl₃) δ: 1.19(t, 3H), 2.58 (q, 2H), 4.57 (s, 2H), 4.60 (s, 2H), 7.30-7.40 (m, 5H)

LRMS: m/z APCI-343 [M+H]+Microanalysis: C₁₃H₁₅₁N₂O requires (%): C, 45.77; H, 4.54; N, 8.01; found (%): C, 45.63; H, 4.42; N, 8.19.

### Preparation 13

### 2-Benzyloxymethyl-1,4-diethyl-5-iodo-1H-imidazole

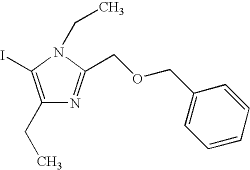

Potassium carbonate (1.21 g, 8.77 mmol) was added to a solution of the compound of preparation 12 (2 g, 5.84 mmol) in DMF (50 mL) and the mixture was stirred at rt for 5 min. Ethyl iodide (514 μL, 6.43 mmol) was then added dropwise to the mixture and stirring continued for 18 h. The mixture was then partitioned between EtOAc (10 mL) and water (100 mL) and the layers were separated. The aqueous solution was extracted with EtOAc (2×100 mL) and the combined organic fraction was washed with brine (100 mL), dried over magnesium sulfate and concentrated in vacuo. The residue was purified by column chromatography on silica gel, eluting with toluene:ethyl acetate 80:20 to afford the title compound (eluted second from the column) as a pale yellow oil in 42% yield (900 mg).

¹H NMR (400 MHz, CDCl₃) δ: 1.15(t, 3H), 1.28 (t, 3H), 2.51 (q, 2H), 4.02 (q, 2H), 4.53 (s, 2H), 4.60 (s, 2H), 7.25-7.34 (m, 5H). LRMS: m/z APCI-371 [M+H]⁺

### Preparation 14

### (1,4-Diethyl-5-iodo-1H-imidazol-2-yl)-methanol

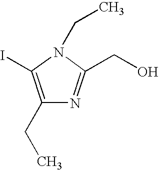

Boron trichloride-methyl sulfide complex solution (2M in DCM, 2.4 mL, 4.80 mmol) was added dropwise to a solution of the compound of preparation 13 (880 mg, 2.38 mmol) in DCM (15 mL) and the mixture was stirred for 4 h at rt. A further amount of boron trichloride-methyl sulfide complex solution (2M in DCM, 1.2 mL, 2.4 mmol) was then added to the reaction mixture and stirring continued for 2 h. The mixture was then diluted with DCM (20 mL) and quenched with sodium hydrogen carbonate solution (30 mL). The layers were separated and the aqueous solution was extracted with DCM (2×30 mL). The combined organic solutions were washed with brine (20 mL), dried over magnesium sulfate and concentrated in vacuo to give a brown oil. The oil was purified by column chromatography on silica gel, eluting with pentane:ethyl acetate 66:33 to 0:100, followed by trituration with diethyl ether/pentane to afford the title compound as a pale brown solid in 75% yield (500 mg).

¹H NMR (400 MHz, CD₃OD) δ: 1.14(t, 3H), 1.35 (t, 3H), 2.50 (q, 2H), 4.10 (q, 2H), 4.64 (s, 2H). LRMS: m/z APCI-281 [M+H]⁺

### Preparation 15

### 2-(1,4-Diethyl-5-iodo-1H-imidazol-2-ylmethyl)-isoindole-1,3-dione

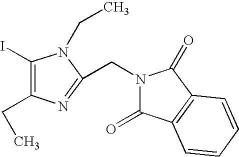

Diisopropyl azodicarboxylate (139 μL, 0.72 mmol) was added to an ice-cooled mixture of the compound of preparation 14 (150 mg, 0.54 mmol), triphenylphosphine (210 mg, 0.80 mmol) and phthalimide (118 mg, 0.80 mmol) in THF (5 mL) and the suspension was stirred at rt for 18 h. The mixture was then concentrated in vacuo and the residue was purified by column chromatography on silica gel, eluting with pentane:ethyl acetate 33:66 to afford the title compound as a pale yellow solid in 36% yield (80 mg).

¹H NMR (400 MHz, CD₃OD) δ: 1.07(t, 3H), 1.33 (t, 3H), 2.45 (q, 2H), 4.16 (q, 2H), 4.97 (s, 2H), 7.82 (m, 2H), 7.89 (m, 2H). LRMS: m/z APCI-410 [M+H]⁺

### Preparation 16

### 3-Chloro-5-{2-[2-(1,3-dioxo-1,3-dihydro-isoindol-2-yl)-ethyl]-3,5-diethyl-3H-imidazol-4-ylsulfanyl}-benzonitrile

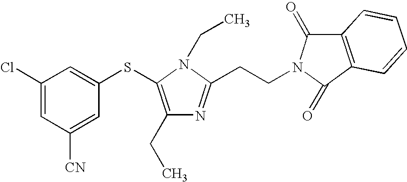

The title compound was prepared from the compound of example 2, using a method similar to that of preparation 15, as a white solid in 65% yield.

¹H NMR (400 MHz, CDCl₃) δ: 0.95(m, 3H), 1.25 (t, 3H), 2.45 (m, 2H), 3.15 (m, 2H), 3.95 (q, 2H), 4.10 (t, 2H), 7.25 (s, 1H), 7.28 (s, 1H), 7.40 (s, 1H), 7.70 (m, 2H), 7.88 (m, 2H). LRMS: m/z APCI-465 [M+H]⁺

### Preparation 17

### 3-Chloro-5-[2-(1,3-dioxo-1,3-dihydro-isoindol-2-ylmethyl)-3,5-diethyl-3H-imidazol-4-ylsulfanyl]-benzonitrile

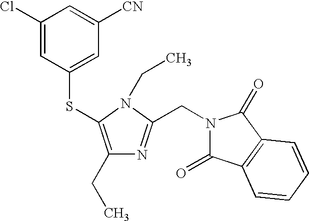

Caesium carbonate (83 mg, 0.26 mmol) was added to a solution of the compound of preparation 6 (37 mg, 0.22 mmol) in MeCN (2 mL) and the mixture was stirred for 10 min. The compound of preparation 15 (70 mg, 0.17 mmol) and copper (I) iodide (10 mg, 0.05 mmol) were then added and the mixture was heated under reflux for 18 h. The reaction mixture was cooled to rt, concentrated in vacuo and partitioned between EtOAc (15 mL) and water (15 mL). The organic layer was separated and the aqueous solution was extracted with EtOAc (3×10 mL). The combined organic solution was washed with brine (15 mL), dried over magnesium sulfate and concentrated in vacuo to give a dark yellow oil. The oil was purified by column chromatography on silica gel, eluting with ethyl acetate:pentane, 50:50 to 67:33 to 100:0, followed by trituration in diethyl ether:pentane to afford the title compound as a solid in 33% yield (25 mg).

¹H NMR (400 MHz, CD₃OD) δ: 1.08(t, 3H), 1.27 (t, 3H), 2.56 (q, 2H), 4.16 (q, 2H), 5.01 (s, 2H), 7.23 (t, 1H), 7.27 (t, 1H), 7.60 (t, 1H) 7.83 (m, 2H), 7.91 (m, 2H). LRMS: m/z APCI-451 [M+H]⁺

### Preparation 18

### 2-Benzyloxymethyl-4-trifluoromethyl-1H-imidazole

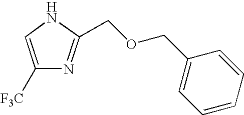

1,1,1-Trifluoro-3,3-dibromoacetone (10.4 mL, 55 mmol) was added to a solution of sodium acetate trihydrate (13.6 g, 100 mmol) in water (45 mL) and the mixture was heated at reflux for 30 min. The mixture was then cooled to rt and added to a solution of benzyloxyacetaldehyde (7.0 mL, 50 mmol) in MeOH (230 mL) and 0.88 ammonia (57 mL), and the mixture was stirred at rt for 18 h. The reaction mixture was concentrated in vacuo to low volume (60 mL), diluted with water (50 mL) and triturated. The resulting precipitate was filtered off and dried in vacuo at 60° C. to afford the title compound as a pale brown solid in 92% yield (13 g).

¹H NMR (400 MHz, DMSO-d₆) δ: 4.51 (s, 2H), 4.52 (s, 2H), 7.25-7.37 (m, 5H), 7.73-7.71 (m, 1H), 12.81 (brs, 1H). LRMS: m/z APCI-257 [M+H]+Microanalysis: C₁₂H₁₁N₂OF₃ requires (%): C, 56.25; H, 4.33; N, 10.93; found (%): C, 56.12; H, 4.29; N, 10.90.

### Preparation 19

### 2-Benzyloxymethyl-5-iodo-4-trifluoromethyl-1H-imidazole

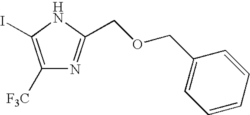

Iodine (12.0 g, 47.5 mmol), periodic acid dihydrate (10.3 g, 45 mmol) and chloroform (45 mL) were added to a solution of the compound of preparation 18 (11.5 g, 45 mmol) in AcOH (135 mL), and the mixture was heated at 60° C. for 4 h. The mixture was then allowed to cool to rt and was poured onto ice-cold 10% aqueous sodium bisulphite solution (600 mL). The aqueous solution was extracted with EtOAc (3×400 mL) and the combined organic solution was washed with brine (400 mL), dried over magnesium sulphate and concentrated in vacuo. Purification of the residue by column chromatography on silica gel, eluting with ethyl acetate:pentane, 33:66, followed by trituration with pentane afforded the title compound as a white powder in 81% yield (14 g).

¹H NMR (400 MHz, DMSO-D₆) δ: 4.55(s, 2H), 4.57 (s, 2H), 7.24-7.36 (m, 5H). LRMS: m/z APCI-383 [M+H]⁺

### Preparation 20

### 3-[2-(2-Benzyloxymethyl)-5-trifluoromethyl-3H-imidazol-4-ylsulfanyl]-5-chloro-benzonitrile

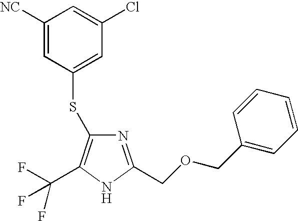

Caesium carbonate (5.73 g, 17.6 mmol) was added to a stirred solution of the compound of preparation 6 (2.71 g, 16.0 mmol) in MeCN (100 mL) and the mixture was stirred for 15 min at rt. A solution of the compound of preparation 19 (6.11 g, 16.0 mmol) in MeCN (100 mL) was then added dropwise, followed by copper (I) iodide (910 mg, 4.8 mmol) and the reaction mixture was heated under reflux for 18 h. After this time, tlc analysis showed that starting material (SM) still remained and so further copper (I) iodide (300 mg, 1.6 mmol) was added to the mixture and heating continued for 30 h. The mixture was then cooled to rt and was concentrated in vacuo. The residue was partitioned between EtOAc (100 mL) and water (100 mL) and the resulting precipitate was filtered off. The layers were separated and the aqueous solution was extracted with EtOAc (2×100 mL). The combined organic solutions were washed with brine (100 mL), dried over magnesium sulfate and concentrated in vacuo to give a green foam. Purification of the foam by column chromatography on silica gel, eluting with toluene:ethyl acetate, 17:83, afforded the title compound in 41% yield (2.8 g) as a 6:1 mixture of product:unreacted SM, respectively.

LRMS: m/z APCI-424 [M+H]⁺

### Preparation 21

### 3-[2-(2-Benzyloxymethyl)-3-ethyl-5-trifluoromethyl-3H-imidazol-4-ylsulfanyl]-5 chloro-benzonitrile

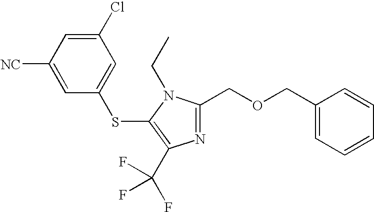

The title compound was prepared from the compound of preparation 20 and ethyl iodide, using a similar method to preparation 9, as a colourless oil in 49% yield.

LRMS: m/z APCI-452 [M+H]⁺

### EXAMPLE 1

### 3-Chloro-5-[3-ethyl-2-(2-hydroxy-ethyl)-5-trifluoromethyl-3H-imidazol-4-ylsulfanyl]-benzonitrile

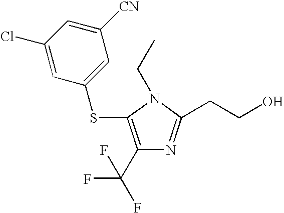

Boron trichloride-methyl sulfide complex solution (2M in DCM, 0.71 mL, 1.42 mmol) was added to a solution of the compound of preparation 9 (330 mg, 0.71 mmol) in DCM (7 mL) and the mixture was stirred for 2 h at rt. The reaction mixture was then basified with sodium hydrogen carbonate solution and stirred for a further 15 min. The mixture was diluted with DCM and the organic layer was separated, dried over magnesium sulfate and concentrated in vacuo. Trituration of the residue with pentane/diisopropyl ether then afforded the title compound as a pale brown solid in 60% yield (160 mg).

¹H NMR (400 MHz, CDCl₃) δ: 1.22(t, 3H), 2.95 (t, 2H), 4.00 (q, 2H), 4.15 (t, 2H), 7.15 (s, 1H), 7.23 (m, 1H), 7.42 (m, 1H) LRMS: m/z APCI-375/377 [M+H]+Microanalysis: C₁₅H₁₃F₃ClN₃OS requires (%): C, 47.33; H, 3.45; N, 10.97; found (%) C, 47.49; H, 3.56, N 11.08.

### EXAMPLE 2

### 3-Chloro-5-[3,5-diethyl-2-(2-hydroxy-ethyl)-3H-imidazol-4-ylsulfanyl]-benzonitrile

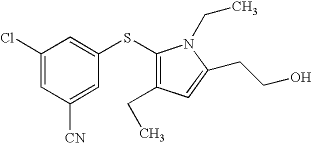

The title compound was prepared from the compound of preparation 10, using a similar method to that of example 1, as pale a brown solid in 61% yield.

¹H NMR (400 MHz, CDCl₃) δ: 1,22(m, 6H), 2.63 (q, 2H), 2.98 (t, 2H), 3.93 (q, 2H), 4.18 (t, 2H), 7.10 (s, 1H), 7.15 (s, 1H), 7.40 (s, 1H) LRMS: m/z APCI-335/337 [M+H]⁺ Microanalysis: C₁₆H₁₈ClN₃OS requires (%): C, 57.22; H, 5.40, N 12.51; found (%) C, 56.92; H, 5.37; N, 12.36

### EXAMPLE 3

### 3-Chloro-5-(3,5-diethyl-2-hydroxymethyl-3H-imidazol-4-ylsulfanyl)-benzonitrile

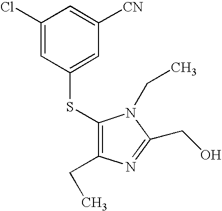

Caesium carbonate (437 mg, 1.34 mmol) was added to a solution of the compound of preparation 6 (227 mg, 1.34 mmol) in MeCN (10 mL) and the mixture was stirred for 10 min. The compound of preparation 14 (250 mg, 0.89 mmol) and copper (I) iodide (51 mg, 0.27 mmol) were then added and the mixture was heated under reflux for 18 h. The reaction mixture was cooled to rt, concentrated in vacuo and partitioned between EtOAc (30 mL) and water (30 mL). The organic layer was separated and the aqueous solution was extracted with EtOAc (3×30 mL). The combined organic solution was washed with brine (30 mL), dried over magnesium sulfate and concentrated in vacuo to give a dark yellow oil. The oil was purified by column chromatography on silica gel, eluting with ethyl acetate, followed by trituration in diethyl ether:pentane to afford the title compound as a white solid in 54% yield (155 mg).

¹H NMR (400 MHz, CD₃OD) δ: 1.17(t, 3H), 1.25 (t, 3H), 2.62 (q, 2H), 4.10 (q, 2H), 4.70 (s, 2H), 7.24 (t, 1H), 7.25 (t, 1H), 7.60 (t, 1H). LRMS: m/z APCI-322 [M+H]⁺

### EXAMPLE 4

### 3-[2-(2-Amino-ethyl)-3,5-diethyl-3H-imidazol-4-ylsulfanyl]-5-chloro-benzonitrile tartrate

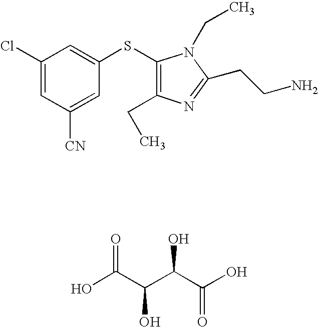

Molecular sieves (4 Å) and hydrazine monohydrate (50 μL, 1 mmol) were added to a suspension of the compound of preparation 16 (95 mg, 0.2 mmol) in EtOH (5 mL) and the mixture was heated at 45° C. for 18 h. Tlc analysis showed that starting material still remained so further hydrazine monohydrate (5 μL, 1 mmol) was added to the mixture and heating continued at 45° C. for 6 h. The reaction mixture was then filtered and the filtrate was washed with sodium sulphite solution, dried over magnesium sulfate and concentrated in vacuo. The residue was purified by column chromatography on silica gel, eluting with dichloromethane:methanol:0.88 ammonia, 95:5:0 to 90:10:1 to give a colourless gum. The gum was then re-dissolved in EtOAc and a solution of L(+) tartaric acid (18 mg, 0.2 mmol) in EtOAc was added. The mixture was concentrated in vacuo and the residue was triturated with ethyl acetate/pentane to afford the title compound as a white solid in 68% yield (48 mg).

¹H NMR (400 MHz, DMSO-D₆) δ: 1.11(m, 6H), 2.50 (m, 2H), 2.98 (m, 2H), 3.20 (m, 2H), 3.75 (bs, 2H), 3.84 (q, 2H), 7.33 (d, 1H), 7.38 (d, 1H), 7.85 (s, 1H).

### EXAMPLE 5

### 3-(2-Aminomethyl-3,5-diethyl-3H-imidazol-4-ylsulfanyl)-5-chloro-benzonitrile

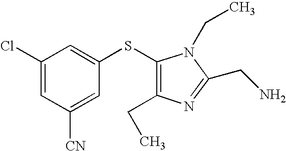

The title compound was prepared as a free amine from the compound of preparation 17, using a method similar to that of example 4 as a colourless oil in 78% yield.

¹H NMR (400 MHz, CD₃OD) δ: 1.18(t, 3H), 1.20 (t, 3H), 2.63 (q, 2H), 3.92 (s, 2H), 4.02 (q, 2H), 7.26 (d, 2H), 7.60 (t, 1H). LRMS: m/z APCI-321 [M+H]⁺

### EXAMPLE 6

### Carbamic acid 5-(3-chloro-5-cyano-phenylsulfanyl)-1,4-diethyl-1H-imidazol-2-ylmethyl ester

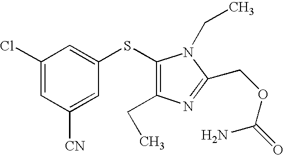

A solution of the compound of example 3 (60 mg, 0.19 mmol) in THF (2 mL) was cooled to 0° C. and treated with trichloroacetylisocyanate (33 μL, 0.27 mmol). The mixture was allowed to warm to rt and was stirred for 3 h. The reaction was then quenched with saturated sodium hydrogen carbonate solution and diluted with DCM (10 mL). The aqueous layer was separated and extracted with DCM (2×10 mL) and the combined organic solution was washed with brine and concentrated in vacuo to low volume. The residue was poured onto a pad of alumina (Brockmann I, neutral alumina treated with 3% w/w water and stirred for 4 days) and left to stand for 15 min. The alumina was then flushed with a mixture of ethyl acetate:methanol, 100:0 to 90:10, and the filtrate was concentrated in vacuo to give a colourless oil. Trituration of the oil with pentane:ethyl acetate afforded the title compound as a white solid in 58% yield (40 mg).

¹H NMR (400 MHz, CD₃OD) δ: 1.17(t, 3H), 1.23 (t, 3H), 2.63 (q, 2H), 4.08 (q, 2H), 5.17 (s, 2H), 7.23 (m, 1H), 7.26 (m, 1H), 7.61 (m, 1H) LRMS: m/z APCI-365 [M+H]⁺

### EXAMPLE 7

### 3-Chloro-5-[3-ethyl-2-(2-hydroxymethyl)-5-trifluoromethyl-3H-imidazol-4-ylsulfanyl]-benzonitrile

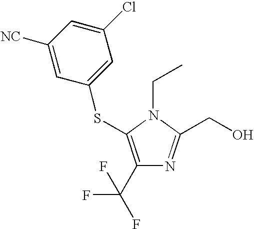

The title compound was prepared from the compound of preparation 21, using a method similar to that of example 1, as a beige solid in 39% yield.

¹H NMR (400 MHz, CDCl₃) δ: 1.30(t, 3H), 4.14 (q, 2H), 4.84 (s, 2H), 7.17 (t, 1H), 7.26 (t, 1H), 7.46 (t, 1H). LRMS: m/z APCI-362 [M+H]⁺

### EXAMPLE 8

### Carbamic acid 5-(3-chloro-5-cyano-phenylsulfanyl)-1-ethyl-4-trifluoromethyl-1H-imidazol-2-ylmethyl ester

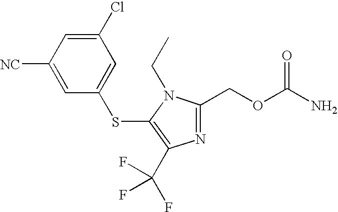

The title compound was prepared from the compound of example 7, using a method similar to that of example 6, as a white solid in 70% yield.

¹H NMR (400 MHz, CDCl₃) δ: 1.26(t, 3H), 4.13 (q, 2H), 4.79 (br s, 2H), 5.26 (s, 2H), 7.13 (t, 1H), 7.25 (t, 1H), 7.46 (t, 1H). LRMS: m/z APCI-404 [M+H]⁺ Microanalysis: C₁₅H₁₂F₃ClN₄O₂S requires (%): C, 44.51; H, 2.99; N, 13.84; found (%) C, 44.45; H, 2.97; N, 13.74.

### Biological Data

The activity of the compounds of the invention as reverse transcriptase inhibitors may be measured using the following assay.

### Inhibition of HIV-1 Reverse Transcriptase Enzyme

The reverse transcriptase activity of the compounds of the invention may be assayed as follows. Using the purified recombinant HIV-1 reverse transcriptase (RT, EC, 2.7.7.49) obtained by expression in Escherichia Coli, a 384-well plate assay system was established for assaying a large number of samples using the [3H]-Flashplate enzyme assay system (NEN-SMP 410A) following the manufacturer's recommendations. The compounds were dissolved in 100% DMSO and diluted with the appropriate buffer to a 5% final DMSO concentration. The inhibitory activity was expressed in percent inhibition relative to the DMSO control. The concentration at which the compound inhibited the reverse transcriptase by 50% was expressed as the IC₅₀ of the compound.

All the Examples of the invention have IC₅₀ values, according to the above method, of less than 1 μM.

1.-10. (canceled)

11. A compound of formula (I): 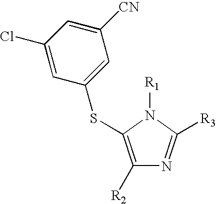
or a pharmaceutically acceptable salt or solvate or derivative thereof, wherein:
R₁ is (C₄-C₄)alkyl or (C₃-C₅)cycloalkyl, wherein said alkyl is optionally substituted by pyridyl or pyridyl N-oxide;
R₂ is (C₁-C₄)alkyl, (C₃-C₆)cycloalkyl, or trifluoromethyl;
R₃ is —(CH₂)ₘOH, —(CH₂)ₘOC(O)NR₄R₅, —(CH₂)ₘNR₄R₅, or —(CH₂)ₘNHC(O)NR₄R₅;
R₄ and R₅ independently are H or (C₁-C₄)alkyl;
m is 1, 2, 3 or 4.

12. A compound according to claim 1, wherein R₄ is (C₁-C₄)alkyl.

13. A compound according to claim 1, wherein R₄ is (C1%)alkyl or trifluoromethyl.

14. A compound according to claim 1, wherein R₃ is —(CH₂)ₘOH, —(CH₂)ₘOC(O)NR₄R₅, or —(CH₂)ₘNR₄R₅.

15. A compound according to claim 1, wherein R₄ and R₅ are H.

16. A compound according to claim 1, wherein m is 1 or 2.

17. A pharmaceutical composition comprising a compound of claim 1, or a pharmaceutically acceptable salt thereof together with one or more pharmaceutically acceptable excipients, diluents or carriers.

18. A pharmaceutical composition according to claim 1 including one or more additional therapeutic agents.

19. A method of treatment of a mammal with a reverse transcriptase inhibitor or modulator, which comprises treating said mammal with an effective amount of a compound of claim 1 or a pharmaceutically acceptable salt thereof.

20. A method of treatment of a mammal with a reverse transcriptase inhibitor or modulator, which comprises treating said mammal with an effective amount of a compound of claim 1 or a pharmaceutically acceptable salt thereof.
